(19) United States
(12) Patent Application Publication
(10) Pub. No.: US 2025/0065055 A1
(43) Pub. Date: Feb. 27, 2025

(54) Injection Needle with Surface Texture Modification and Method of Manufacturing Thereof

(71) Applicant: Becton, Dickinson and Company, Franklin Lakes, NJ (US)

(72) Inventors: Thomas Devouassoux, Saint Martin d'Uriage (FR); Christopher R. Maul, Belton, TX (US); Jacques Moret, St Martin le Vinoux (FR)

(21) Appl. No.: 18/455,004

(22) Filed: Aug. 24, 2023

(51) Int. Cl. A61M 5/32

(52) U.S. Cl. CPC A61M 5/32; A61M 2207/10

(57) ABSTRACT

Provided herein is a method for modifying a surface texturing of an injection needle. The method includes positioning one or more injection needles onto a support tool, each of the one or more injection needles comprising a cylindrical needle body having a distal end and a proximal end and defining a lumen therein. The method also includes directing lasers from one or more laser sources onto an outer surface of the needle body of each of the one or more injection needles. The lasers form a pattern of depressions in the outer surface of the needle body of each of the one or more injection needles, to form a textured outer surface on the one or more injection needles.

### BACKGROUND OF THE INVENTION

### Field of the Invention

The present disclosure relates generally to needles for use in medical injection devices and, more particularly, to needles having a modified surface texture and a method of manufacturing thereof.

### Description of Related Art

Medical injection devices, such as syringes, are used in a variety of environments for administering liquids (e.g., medications or drugs) to a patient. Syringes will typically include a syringe barrel with a plunger assembly inserted through an open proximal end of the barrel and an opening at the opposite distal end adapted to receive a needle therein by which a fluid is injected into the patient.

In manufacturing of the syringe, a method is required for securing the needle within the opening at the distal end of the syringe barrel. In some manufacturing techniques, each of the needle and the syringe barrel are separately formed/manufactured and the needle is subsequently secured within the opening at the distal end of the syringe barrel via a UV-cured glue or other adhesive. In other manufacturing techniques, the syringe barrel may be formed in a manner by which it secures the needle thereto during manufacturing. If the syringe barrel is formed of a polymeric material, the syringe barrel may be over-molded onto the needle so as to secure the needle within the opening at the distal end of the syringe barrel. If the syringe barrel is formed of glass, the syringe barrel may be glass-formed onto the needle so as to secure the needle within the opening at the distal end of the syringe barrel.

In any of the manufacturing techniques described above, it is desirable to provide a surface texturing or “rugosity” to at least the portion of the needle that is secured to the syringe barrel, so as to provide for improved adhesion/bonding between the needle and the syringe barrel. This surface texturing of the needle is commonly provided by performing sand-blasting on the needle. However, it is recognized that sand-blasting may be difficult to apply to small size needles, along with being a time-consuming process. Furthermore, it is recognized that sand-blasting may lack accuracy when forming the surface texturing, both in controlling the area to which surface texturing is applied and/or in forming particular pattern of surface texturing.

Accordingly, a need exists in the art for a process by which surface texturing or rugosity may be provided on an injection needle, with the surface texturing or rugosity providing improved adhesion and bonding between the needle and its support, and thus a better resistance to pull out force.

### SUMMARY OF THE INVENTION

Provided herein is a method for modifying a surface texturing of an injection needle. The method includes positioning one or more injection needles onto a support tool, each of the one or more injection needles comprising a cylindrical needle body having a distal end and a proximal end and defining a lumen therein. The method also includes directing lasers from one or more laser sources onto an outer surface of the needle body of each of the one or more injection needles. The lasers form a pattern of depressions in the outer surface of the needle body of each of the one or more injection needles, to form a textured outer surface on the one or more injection needles.

In certain configurations, the lasers are directed onto the outer surface of the needle body at the proximal end thereof.

In certain configurations, in directing the lasers onto the outer surface of the needle body of each of the one or more injection needles, the lasers are directed to a plurality of distinct locations to form the pattern of depressions.

In certain configurations, in directing the lasers onto the outer surface of the needle body of each of the one or more injection needles, the lasers are directed to each of the plurality of distinct locations a number of times, to provide a repetitive laser application pattern.

In certain configurations, the pattern of depressions comprises a plurality of craters, and wherein a depth of each of the plurality of craters is controlled based on the number of times the lasers are directed to each of the plurality of craters.

In certain configurations, the depth of each of the plurality of craters is between 1.5 and 9.0 micrometers.

In certain configurations, each of the plurality of craters has a rounded bottom and a sharp edge about the circumference thereof.

In certain configurations, each of the plurality of craters is 30 micrometers in diameter.

In certain configurations, the pattern of depressions comprises a plurality of rows, with each of the plurality of rows comprising a plurality of depressions

In certain configurations, each of the plurality of rows extends circumferentially about the outer surface to cover a half cylinder of the cylindrical outer body.

In certain configurations, the method further comprises reversing an orientation of each of the one or more injection needles, and wherein each of the plurality of rows extends circumferentially about an entirety of the outer surface to cover a full cylinder of the cylindrical outer body.

In certain configurations, the lasers comprise ultraviolet lasers.

Also provided herein is an injection needle that includes a cylindrical needle body comprising a distal end and a proximal end and defining a lumen, and a beveled injection tip formed at the distal end. The proximal end of the cylindrical needle body includes a textured outer surface comprising a plurality of rows, each or the plurality of rows having a plurality of craters depressed radially inward into the outer surface, with the plurality of rows arranged axially along a length of the cylindrical needle body at the proximal end. The plurality of craters are configured to enhance bonding of the textured outer surface to a surrounding needle support.

In certain configurations, each of the plurality of rows is immediately adjacent another of the plurality of rows.

In certain configurations, the plurality of rows comprises at least a first row grouping and a second row grouping, each of the first row grouping and the second row grouping including a number of rows, and wherein the first row grouping is spaced apart axially from the second row grouping by a gap where an outer surface of the cylindrical needle body is free of craters.

In certain configurations, each of the plurality of craters has a rounded bottom and a sharp edge about the circumference thereof.

In certain configurations, the depth of each of the plurality of craters is between 1.5 and 9.0 micrometers.

In certain configurations, each of the plurality of craters is 30 micrometers in diameter.

In certain configurations, each of the plurality of rows extends circumferentially about the outer surface to cover a half cylinder of the cylindrical outer body.

In certain configurations, each of the plurality of rows extends circumferentially about an entirety of the outer surface to cover a full cylinder of the cylindrical outer body.

### BRIEF DESCRIPTION OF THE DRAWINGS

FIG. 1 is an exploded view of a syringe, with which embodiments of the disclosure may be implemented;

FIG. 2 is a cross-section view taken along line 2-2 of FIG. 1, showing the needle retained within the hub of the syringe barrel;

FIG. 3 is a perspective view of an apparatus for modifying a surface texturing of an injection needle, according to an aspect of the disclosure;

FIG. 4 is a schematic view of a texturing tool included in the apparatus of FIG. 3, directing lasers onto a plurality of needles to modify the surface texturing thereof, according to an aspect of the disclosure;

FIG. 5 is a perspective view of a support plate of the texturing tool included in the apparatus of FIG. 3 and needles retained therein, in a first orientation;

FIG. 6 is a perspective view of a support plate of the texturing tool included in the apparatus of FIG. 3 and needles retained therein, in a second orientation;

FIG. 7 illustrates a pattern of depressions formed on the outer surface of a needle, according to an aspect of the disclosure;

FIG. 8 illustrates a pattern of depressions formed on the outer surface of a needle, according to another aspect of the disclosure;

FIG. 9 is a cross-sectional view of a needling, illustrating a diameter and depth of depressions formed on the outer surface of a needle, according to an aspect of the disclosure; and

FIG. 10 is a cross-sectional view of a needling, illustrating a diameter and depth of depressions formed on the outer surface of a needle, according to another aspect of the disclosure.

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary embodiments of the invention. Hence, specific dimensions and other physical characteristics related to the embodiments disclosed herein are not to be considered as limiting.

In the present disclosure, the distal end of a component or of a device means the end furthest away from the hand of the user and the proximal end means the end closest to the hand of the user, when the component or device is in the use position, i.e., when the user is holding a syringe in preparation for or during use. Similarly, in this application, the terms “in the distal direction” and “distally” mean in the direction toward the distal tip of the syringe, and the terms “in the proximal direction” and “proximally” mean in the direction opposite the direction of the distal tip of the syringe.

Aspects and embodiments of the disclosure are directed to a method for modifying a surface texturing or rugosity of an injection needle, with the surface texturing or rugosity providing improved adhesion and bonding between the needle and its support, and thus a better resistance to pull out force.

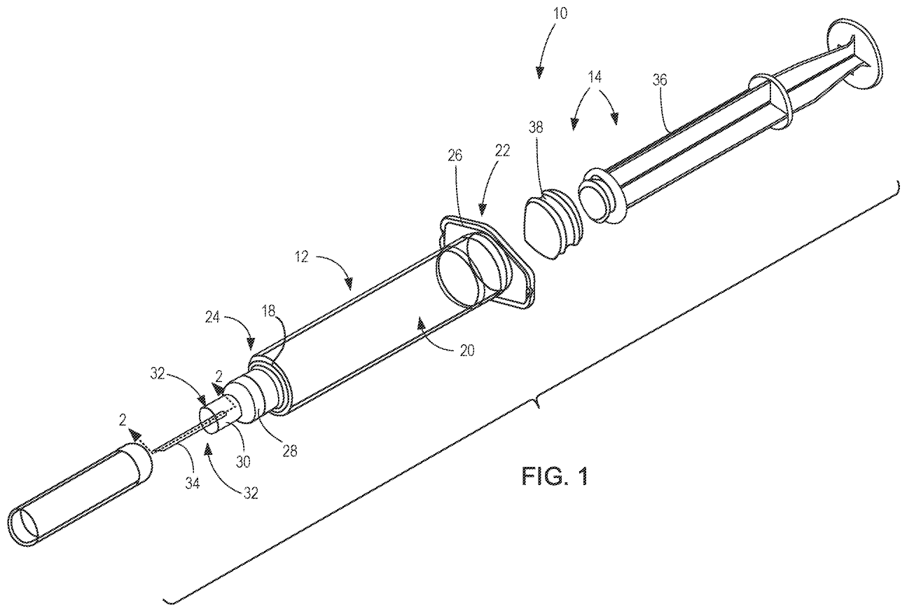

Referring to FIG. 1, shown is a non-limiting embodiment of a medical injection device with which aspects or embodiments of the disclosure may be implemented. While the medical injection device is shown and described hereafter as a syringe (“syringe 10”), it is recognized that other medical injection devices may incorporate aspects of the disclosure, as described in detail here below.

As shown in FIG. 1, the syringe 10 generally includes a syringe barrel 12 and a plunger assembly 14 (including a rod 36 and stopper 38). The plunger assembly 14 is movable within the syringe barrel 12 along a longitudinal axis to an advanced position to facilitate administering of an injectable fluid (e.g., medication) to a patient, for example. The syringe barrel 12 is formed of a generally cylindrical outer wall 16 and an end member 18 that collectively define a chamber 20 for retaining fluid therein. The syringe barrel 12 includes an open proximal end 22 configured to receive the plunger assembly 14 therein and a distal end 24 at which end member 18 is positioned. The proximal end 22 of the syringe barrel 12 may include a flange 26 to facilitate handling and positioning of the syringe 10 and to maintain the relative position of the syringe barrel 12 with respect to the plunger assembly 14 during medication administration. At the distal end 24, the end member 18 may include a shoulder 28 which narrows with respect to the cylindrical outer wall 16, as well as a hub portion 30 extending out distally from shoulder 28. The hub portion 30 is formed as a partially hollow member that defines a channel 32 therethrough in fluid communication with the chamber 20. A needle 34 is attached to the hub portion 30 within channel 32, i.e., a staked needle, such as by being glued or otherwise secured to the hub portion 30.

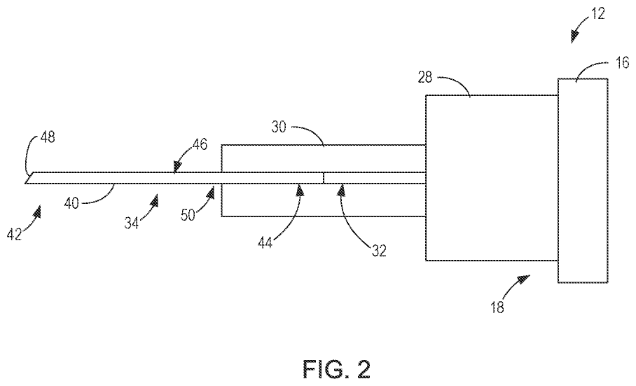

Securement of the needle 34 within hub is shown in further detail in FIG. 2. As shown therein, needle 34 is formed of a cylindrical body 40 comprising a distal end 42 and a proximal end 44, with the body 40 defining a lumen 46 that extends a length of the needle 34 from the distal end 42 to the proximal end 44. The distal end 42 of the needle 34 includes a beveled injection tip 48 formed thereat that provides for injection of the needle 34 into a patient during use of the syringe 10. The proximal end 44 of needle 34 is held within channel 32 of hub 30 so as to retain the needle 34 securely in place during use of the syringe 10. In some embodiments, the needle 34 is secured within channel 32 via glue or another suitable adhesive, with UV-cured glue being a primary example. In other embodiments, the needle 34 is secured within channel 32 via an over-molding of the syringe barrel 12 (i.e., of hub 30) onto the needle 34 (when the syringe barrel 12 is composed of a polymeric material) or via a glass-forming of the syringe barrel 12 (i.e., of hub 30) onto the needle 34 (when the syringe barrel 12 is composed of glass).

According to aspects of the disclosure, it is desirable to configure at least a portion of the needle 34, such as the proximal end 44 thereof, so that the outer surface 50 (of body 40) is textured to have a rugosity that can aid in securing the needle 34 within hub 30. The textured outer surface 50 provides for enhanced bonding between the needle 34 and hub 30 of syringe barrel 12. whether the needle 34 is glued to hub 30 or whether the hub 30 is over-molded or glass-formed onto the needle 34. As described in detail here below, a method for modifying a surface texturing of an injection needle 34 is provided, with the method producing a needle 34 having a desired pattern of depressions in the outer surface 50 thereof to provide the textured outer surface.

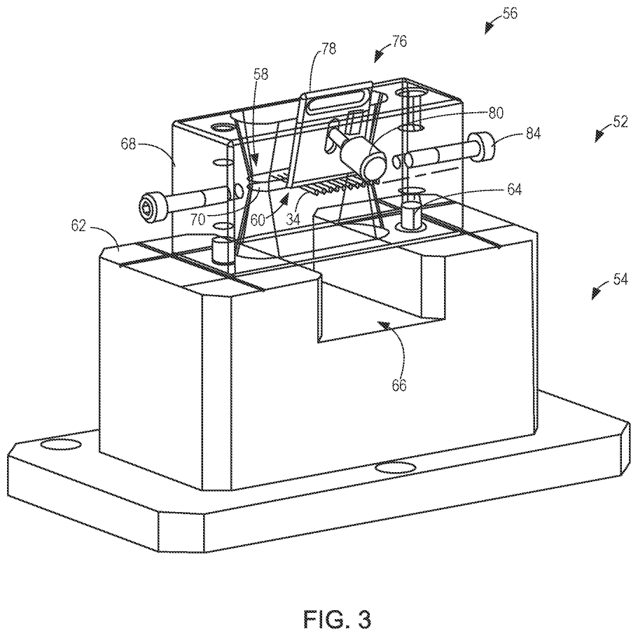

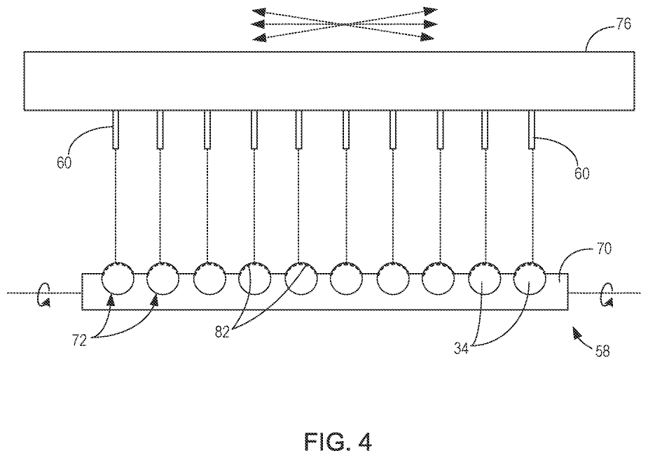

Referring now to FIGS. 3-6, an apparatus 52 that may be used to modify a surface texturing of an injection needle 34 is provided in accordance with an aspect of the disclosure. The apparatus 52 may comprise a base 54 and a texturing tool 56 provided on the base 54. In one embodiment, the texturing tool 56 includes a securing or locking unit 58 within which one or more needles 34 may be secured for processing, along with one or more laser emitters or sources 60 configured to direct lasers toward the needles 34 to provide texturing thereto.

Each of the base 54 and locking unit 58 may be formed of aluminum or another suitable material. The base 54 may be configured to provide a raised surface or platform 62 onto which locking unit 58 may be secured, such as via alignment pins 64, with a recessed central area 66 in which laser processing of the needles 34 may be performed. The locking unit 58 may include an outer housing 68 mounted to base 54, along with a support plate 70 positioned within housing 68 that is generally positioned over the recessed central area 66 of base 54, with the support plate 70 having a plurality of grooves 72 within which the needles 34 may be placed. In the illustrated embodiment, the locking unit 58 is configured to retain ten (10) needles 34 therein for surface texturing, but it is recognized that a greater or lesser number of needles 34 could be retained thereby. The needles 34 may be secured to locking unit 58 such that the portion of the needles 34 to be processed, i.e., the proximal ends 44 thereof, are positioned over an opening 74 provided in the support plate 70.

The one or more laser sources 60 included in texturing tool 56 are configured to direct lasers toward the needles 34 to provide texturing thereto. In some embodiments, the laser sources 60 generate ultraviolet (UV) lasers for processing of the needles 34. The laser source(s) 60 may be mounted on texturing tool 56 (i.e., on housing 68 thereof) by a laser guiding structure 76 that includes a mount 78 and an actuator 80 (e.g., electric motor), with the guiding structure 76 enabling movement (i.e., linear movement and/or rotational movement) of the laser sources 60, so as to provide for a desired positioning/orientation of the laser sources 60 relative to the needles 34 held on support plate 70. Via controlling of the positioning/orientation of the laser sources 60, lasers may thus be directed toward specified locations on the needles 34 to form a desired pattern of depressions 82 in the outer surface 50 of the needles 34.

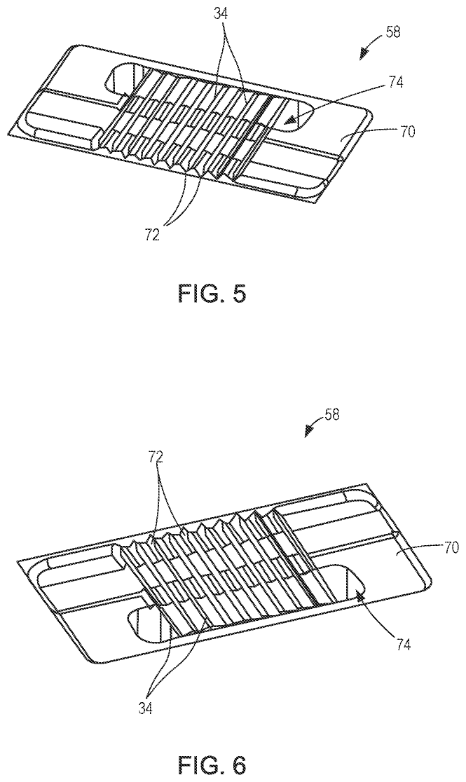

In some embodiments, the texturing tool 56 may further include a rotational shaft 84 that provides for rotation of the support plate 70 (and needles 34) relative to the laser source(s) 60. Via turning of the rotational shaft 84, the support plate 70 and needles 34 may be reoriented such that a bottom surface of needles 34 (i.e., a bottom half cylinder portion of the proximal end 44 of needles 34) may be processed to have surface texturing thereon. That is, it is recognized that if needles 34 and support plate 70 are left in a stationary position, the laser sources 60 are able to direct lasers to only an upper half cylinder portion of the needles 34 to provide surface texturing thereto, and that turning of rotational shaft 84 provides for reorienting of the support plate 70 and needles 34 (as shown in FIGS. 5 and 6) and enables surface texturing to be provided circumferentially about an entirety of the outer surface 50 of the needles 34 at the proximal end 44 thereof. Accordingly, based on a selective rotation of the support plate 70 and needles 34 provided by rotational shaft 84, surface texturing of the needles 34 may be controlled to form a semi-cylindrical pattern of depressions on only half the cylindrical outer surface 50 of needles 34 (on proximal end 44) or to form an entire cylindrical pattern of depressions circumferentially about the entire cylindrical outer surface 50 of needles 34 (on proximal end 44).

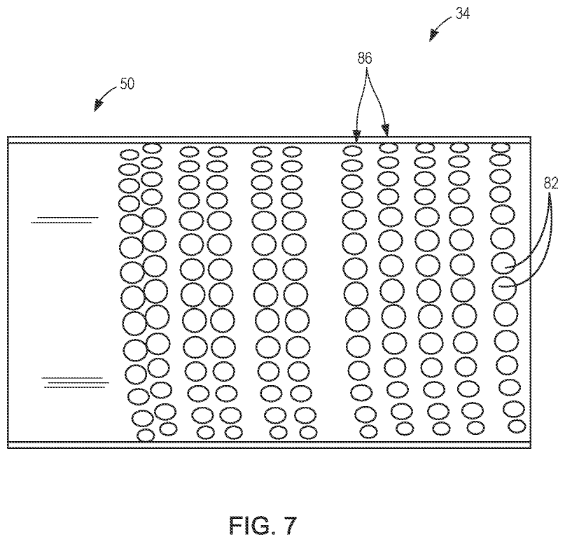

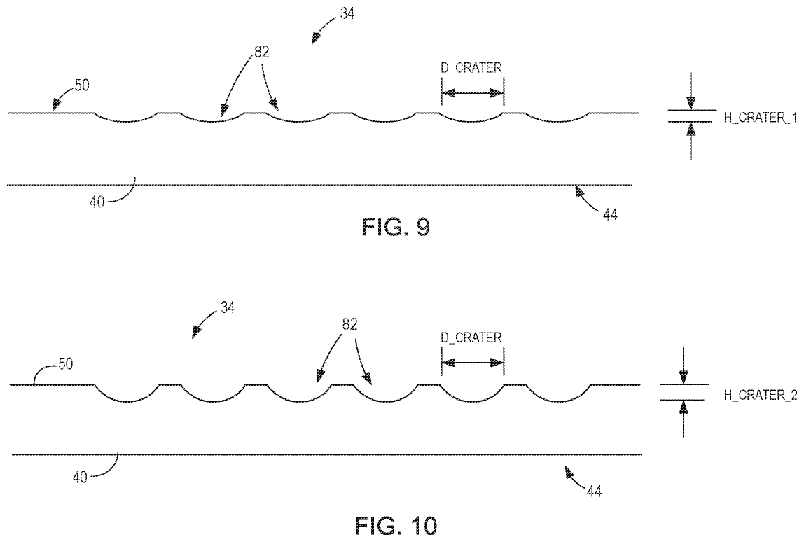

Referring now to FIGS. 7-10, detailed views of the outer surface 50 of a needle 34 are provided according to various aspects of the disclosure, in order to show surface texture patterns that may be formed on a needle 34. As shown therein, in directing the lasers onto the outer surface 50 of the needle 34, the lasers are directed to a plurality of distinct locations to form a pattern of depressions 82 therein. Each of the depressions 82 may be configured as a dot or crater (hereafter “crater 82”) that extends radially inward into the needle 34. As best shown in FIGS. 9 and 10, each of the craters 82 may be configured to have a rounded bottom and a sharp edge about the circumference thereof. The diameter (D_crater) and the depth (H_crater) of the craters 82 may be controlled based on surface texturing considerations and by the configuration and controlling of laser sources 60. Regarding the diameter, D_crater, of each of the plurality of craters 82, the craters 82 may be formed to have a diameter of approximately 30 micrometers, in some embodiments, although diameters of greater or lesser size are possible. Regarding the depth, H_crater, of each of the plurality of craters 82, it is recognized that the depth may be controlled based on the number of passes/exposures of each of the craters 82 to a laser from laser sources 60. That is, the depth of each of craters 82 may be increased by repetitively directing a laser thereto, with each exposure of the crater 82 to a laser removing additional material therefrom, so as to increase the depth of the crater 82. For example, the laser source(s) 60 and laser guiding structure 76 may be controlled so that lasers are directed to each of the craters 82 a number of times, with each of the craters 82 being processed/exposed to a laser between one (1) and four (4) times, according to a non-limiting embodiment. With such singular or repeated laser exposure, the depth of each of the plurality of craters 82 may be controlled to be between approximately 1.5 micrometers (1 laser exposure) and 9.0 micrometers (4 laser exposures).

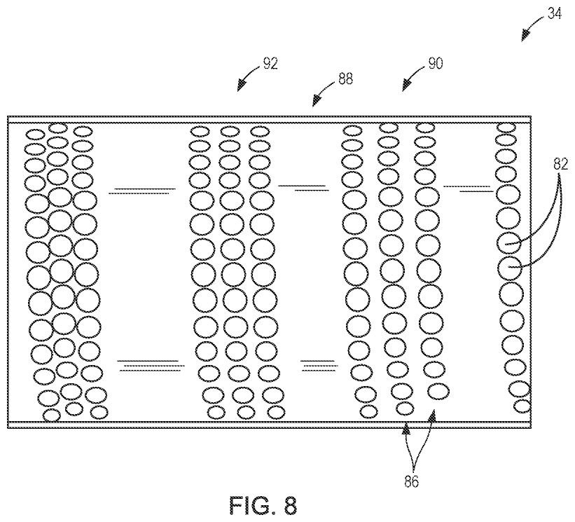

As shown in FIGS. 7 and 8, a pattern of depressions (i.e., craters 82) formed in the outer surface 50 of a needle 34 can vary based on the selective controlling/positioning of the lasers directed onto the needle 34. In each of the embodiments, the craters 82 are arranged/patterned so as to form a plurality of rows 86 that each extend circumferentially about the outer surface 50—with each of the rows 86 of craters 82 covering a half cylinder of the cylindrical outer body 40 or an entirety of the cylindrical outer body 40 (as explained in detail above) at the proximal end 44 of the needle 34. The rows 86 of craters 82 are arranged axially along the needle 34 (along axis A_needle). In some embodiments, the craters 82 are patterned such that each of the plurality of rows 86 of craters 82 is immediately adjacent another of the plurality of rows 86 of craters 82 (i.e., no axial gap between adjacent rows 86 of craters 82), as shown in FIG. 7. In other embodiments, the craters 82 are patterned such that axial gaps 88 are provided between groupings of rows 86 of craters 82, as shown in FIG. 8. That is, the rows 86 of craters 82 may be arranged in groupings—with a first row grouping 90 including a plurality of rows 86 of craters 82 (e.g., 3 rows) and a second row grouping 92 including a plurality of rows 86 of craters 82 (e.g., 3 rows), where the first row grouping 90 is spaced apart axially from the second row grouping 92 by a gap 88 where an outer surface 50 of the cylindrical needle body 40 is free of craters 82.

While certain patterns of depressions are shown in FIGS. 7 and 8, it is recognized that other patterns of depressions 82 could be formed/provided on the outer surface 50 of the needle 34, and that aspects of the disclosure are not limited to those shown and described in detail herein. In general, the pattern of depressions 82 should provide a textured outer surface 59 on the needle 34 that provides for improved adhesion and bonding between the needle 34 and its support within which it is retained (e.g., hub 30 in FIG. 1).

Beneficially, embodiments of the invention thus are directed to a method for modifying a surface texturing of an injection needle to provide the outer surface 50 of the needle with a rugosity that improves adhesion and bonding between the needle and its support. With such surface texturing/rugosity, the needle provides improve resistance to pull out force from it support, whether the needle is adhered within the support or the support is over-molded or glass-formed onto the needle.

Although the present disclosure has been described in detail for the purpose of illustration based on what is currently considered to be the most practical and preferred embodiments or aspects, it is to be understood that such detail is solely for that purpose and that the present disclosure is not limited to the disclosed embodiments or aspects, but, on the contrary, is intended to cover modifications and equivalent arrangements that are within the spirit and scope of the appended claims. For example, it is to be understood that the present disclosure contemplates that, to the extent possible, one or more features of any embodiment may be combined with one or more features of any other embodiment.

The invention claimed is:

1. A method for modifying a surface texturing of an injection needle, the method comprising:
positioning one or more injection needles onto a support tool, each of the one or more injection needles comprising a cylindrical needle body having a distal end and a proximal end and defining a lumen therein; and
directing lasers from one or more laser sources onto an outer surface of the needle body of each of the one or more injection needles;
wherein the lasers form a pattern of depressions in the outer surface of the needle body of each of the one or more injection needles, to form a textured outer surface on the one or more injection needles.

2. The method of claim 1, wherein the lasers are directed onto the outer surface of the needle body at the proximal end thereof.

3. The method of claim 1, wherein in directing the lasers onto the outer surface of the needle body of each of the one or more injection needles, the lasers are directed to a plurality of distinct locations to form the pattern of depressions.

4. The method of claim 3, wherein in directing the lasers onto the outer surface of the needle body of each of the one or more injection needles, the lasers are directed to each of the plurality of distinct locations a number of times, to provide a repetitive laser application pattern.

5. The method of claim 4, wherein the pattern of depressions comprises a plurality of craters, and wherein a depth of each of the plurality of craters is controlled based on the number of times the lasers are directed to each of the plurality of craters.

6. The method of claim 5, wherein the depth of each of the plurality of craters is between 1.5 and 9.0 micrometers.

7. The method of claim 5, wherein each of the plurality of craters has a rounded bottom and a sharp edge about the circumference thereof.

8. The method of claim 5, wherein each of the plurality of craters is 30 micrometers in diameter.

9. The method of claim 1, wherein the pattern of depressions comprises a plurality of rows, with each of the plurality of rows comprising a plurality of depressions.

10. The method of claim 9, wherein each of the plurality of rows extends circumferentially about the outer surface to cover a half cylinder of the cylindrical outer body.

11. The method claim 10, wherein the method further comprises reversing an orientation of each of the one or more injection needles; and
wherein each of the plurality of rows extends circumferentially about an entirety of the outer surface to cover a full cylinder of the cylindrical outer body.

12. The method of claim 1, wherein the lasers comprise ultraviolet lasers.

13. An injection needle comprising:
a cylindrical needle body comprising a distal end and a proximal end and defining a lumen; and
a beveled injection tip formed at the distal end;
wherein the proximal end of the cylindrical needle body comprises a textured outer surface, the textured outer surface comprising a plurality of rows, each or the plurality of rows comprising a plurality of craters depressed radially inward into the outer surface, and wherein the plurality of rows are arranged axially along a length of the cylindrical needle body at the proximal end; and
wherein the plurality of craters is configured to enhance bonding of the textured outer surface to a surrounding needle support.

14. The injection needle of claim 13, wherein each of the plurality of rows is immediately adjacent another of the plurality of rows.

15. The injection needle of claim 13, wherein the plurality of rows comprises at least a first row grouping and a second row grouping, each of the first row grouping and the second row grouping including a number of rows, and wherein the first row grouping is spaced apart axially from the second row grouping by a gap where an outer surface of the cylindrical needle body is free of craters.

16. The injection needle of claim 13, wherein each of the plurality of craters has a rounded bottom and a sharp edge about the circumference thereof.

17. The injection needle of claim 13, wherein the depth of each of the plurality of craters is between 1.5 and 9.0 micrometers.

18. The injection needle of claim 13, wherein each of the plurality of craters is 30 micrometers in diameter.

19. The injection needle of claim 13, wherein each of the plurality of rows extends circumferentially about the outer surface to cover a half cylinder of the cylindrical outer body.

20. The injection needle of claim 13, wherein each of the plurality of rows extends circumferentially about an entirety of the outer surface to cover a full cylinder of the cylindrical outer body.
